(19) United States
(12) Patent Application Publication
(10) Pub. No.: US 2026/0061021 A1
(43) Pub. Date: Mar. 5, 2026

(54) DUAL STAGE HERBAL NUTRACEUTICALS

(71) Applicant: Kokora, Inc., Brevard, NC (US)

(72) Inventor: Richard Peter Scalzo, Brevard, NC (US)

(73) Assignee: Kokora, Inc., Brevard, NC (US)

(21) Appl. No.: 19/313,085

(22) Filed: Aug. 28, 2025

Related U.S. Application Data
(60) Provisional application No. 63/688,506, filed on Aug. 29, 2024.

(51) Int. Cl. A61K 36/9066; A61K 36/67

(52) U.S. Cl. CPC A61K 36/9066; A61K 36/67; A61K 2236/333; A61K 2236/51

(57) ABSTRACT

Pharmaceutical, nutritional, and nutraceutical formulation, and manufacturing process for the treatment and nutrition of individuals with diabetes. This patent relates to a pharmaceutical and nutritional invention for a pharmaceutical, nutraceutical, and nutritional formulation for the treatment and dietary supplementation of individuals with diabetes, as well as the preparation method for both. The pharmaceutical formulation is composed of seed extracts from the species jambolão (Syzygium cumini L), of the Myrtaceae family, and quixaba (Sideroxylon obtusifolium), of the Sapotaceae family. The nutritional formulation is composed of jambolão seed powder and quixaba seed powder. the present invention is in the fields of pharmacy and nutrition, notably where they can be produced for human use in the following pharmaceutical, nutraceutical and food forms: capsules, tablets and solutions, microgranules, nanocapsules, pellets, syrups, suspensions, emulsions, and can also be incorporated into various foods such as cereal bars, biscuits, flours, shakes, among others, not being limited to these food forms presented, but becoming in itself a food fortification agent.

### RELATED APPLICATION

The present application claims priority to U.S. Provisional No. 63/688,506 filed Aug. 29, 2024, the disclosures of which are incorporated herein in their entireties.

### FIELD

The present disclosure relates to a dual stage herbal nutraceutical in which herb-based phytochemicals and phytonutrients are released and absorbed by the body in stages in a dual action, namely, both rapidly and slowly.

### BACKGROUND

Nutraceuticals are bioactive materials that promote and protect health. The word “nutraceutical” combines the words “nutrition” and “pharmaceutical”. One common method of delivering nutraceuticals is in dietary supplements incorporating extracts derived from a wide variety of herbs, which provide an equally wide variety of health benefits.

Herbal materials such as plant-based material and fungi have various phytochemicals and phytonutrients that when extracted may provide health benefits such as preventative action against viruses, bacteria and cancers, may function as antioxidants, stimulate brain development, improve appetite, decrease nausea, reduce inflammation, provide energy, provide immune support, support gut health, and the like. Exemplary phytochemicals and phytonutrients include phenols, terpenes, phytosterols, glycosides, flavinoids and saponins.

Various methods of extracting phytochemicals from herbal materials have been suggested. Exemplary methods include alcohol extraction, precipitation dialysis, crystallization, fractionation, supercritical fluid extraction, membrane extraction, etc. Often multiple extraction methods may be used depending on the nature of active ingredients of the herbal material. For example, the application of heat may be dilatory for some herbal materials. This is particularly a problem when spray drying an alcohol-extracted herbal material. Moreover, such spray-dried extracts when in powder form, may require high levels of maltodextrin needed for processing.

Another concern is that liquid extracts of a herbal material tend to have higher concentrations of the extract, thus people often find them difficult to consume due to an unpleasant taste and high alcohol content.

### SUMMARY

A process for providing a dual stage herbal nutraceutical is disclosed. The steps may include extracting a crude herbal material with a water/alcohol solution to provide an extract separate from the crude herbal material. The alcohol concentration of the crude herbal material extract may be reduced to provide a low alcohol extract. The low alcohol extract is then mixed with a crude herbal powder of the same herb from which the extract was made and blended into the low alcohol extract. The mixture or blend of crude herbal material solids and low alcohol extract may then be freeze-dried. The freeze-dried herbal blend may be blended with an anti-caking agent and may then be encapsulated in a capsule such as in a plant-based capsule. The dual stage nutraceutical may be used to treat a wide variety of health issues in which the dual action of the low alcohol extract portion having a first release profile and the crude herbal material having a second release profile provide a rapid release and a slow release.

### DETAILED DESCRIPTION

The foregoing and other aspects of the present disclosure will now be described in more detail with respect to the description and methodologies provided herein. It should be appreciated that the disclosure can be embodied in different forms and should not be construed as limited to the embodiments set forth herein. Rather, these embodiments are provided so that this disclosure will be thorough and complete, and will fully convey the scope of the disclosure to those skilled in the art.

The terminology used in the description of the disclosure herein is for the purpose of describing particular embodiments only and is not intended to be limiting of the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

All patents, patent applications and publications referred to herein are incorporated by reference in their entirety. In case of a conflict in terminology, the present specification is controlling.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

The term “herbal materials” may include plant materials such as herbs (e.g., oregano and basil), tree barks, roots, seeds, and the like, and non-plant materials like mushrooms (fungi). The crude herbal material may be organic, cultivated or wild. Once harvested, the crude herbal material may be ground, or cut-up prior to extraction. More than one crude herbal material may be extracted together or separately.

Extraction may be conducted with water and an alcohol. The term “water” may include tap water, deionized water, spring water, distilled water or well water. The term “alcohol” may include methanol, ethanol, isopropanol, isobutane, and butanol. In a preferred embodiment, the water may be deionized water and the alcohol ethanol. The alcohol to water ration may typically range from 20:80 to 30:70 alcohol to water. Agitation or other kinetic actions may be utilized to facilitate extraction and release of phytochemicals and phytonutrients from the crude herbal material. Once contacted and extracted with the alcohol/water mixture, the extracted liquid may be separated from the so-called “spent” extracted solid herbal material.

The liquid extract portion may then be concentrated, for example, using low heat and vacuum. The remaining alcohol concentration at this point may be between 25 to 50 percent alcohol. The extract may have an alcohol concentration ratio of 50 to 75 percent in a weight to volume ratio of 1:1 to 1:4. This allows polar and non-polar plant constituents to be extracted. The alcohol concentration of the extract may be further reduced to provide an extract of substantially low alcohol content of less than 5 percent, for example.

The low alcohol extract may then be blended with powdered crude herb material from the same plant as which the extract is made. The mixing of like herb with the liquid extract allows the liquid extract to adhere to the crude herb powder facilitating freeze drying. The also increases the full spectrum of plant nutrient density. Typically the crude powder may be powdered to a 60 to 80 mesh. In one embodiment, the dried extract may be 60 to 90 percent by weight and the crude herb material may be about 10 to 40 percent by weight. The blend of crude herb material and low alcohol extract may be mixed with agitation or other kinetic action followed by freeze drying. In operation, the crude herbal material and low alcohol extract may be placed in a freeze dryer chilled to a freezing temperature, i.e., less than 0° C. and the freeze dryer may be under vacuum. The chilling may be conducted at a rapid cool rate or a slow, controlled rate. After freeze drying, the extract and crude herbal material/alcohol mixture is a powder. An anti-caking agent such as silicon dioxide, stearic acid, or magnesium stearate, for example may be added to the freeze-dried powder.

In an alternate embodiment, the blend of crude herb material and low alcohol extract may compromise more than one crude herbal material and/or more than one low alcohol extract all blended or mixed together prior to freeze drying and forming a powder.

The blend of freeze-dried crude herbal material and low alcohol extract may optionally be dried to a moisture content of less than about 5 percent. The dried blend in powder form may be encapsulated in a vegetable-based capsule such as capsules described, for example, in U.S. Pat. No. 6,238,696 B1, the disclosure of which is incorporated by reference in its entirety. Other suitable forms of herbal delivery include tablets, lozenges, troches, and crystallized extract.

The capsule will have two or more types of herbal materials with active phytochemicals and phytonutrients, namely, the capsule comprises the freeze-dried liquid extract and the crude herbal materials. The liquid extract may be adhered to the surface of the crude herb portion. Once ingested, the low alcohol extract portion will have a first release profile and the crude herb material portion will have a second release profile. In this manner, the extract of freeze-dried blend may release its phytochemicals and phytonutrients for an initial rapid absorption. In contrast, the phytochemicals and phytonutrients of the crude herbal materials may be absorbed and slowly released further down in the digestive tract.

Exemplary but not limited to, crude herbal materials may include Andrographis, Ashwagandha Root and Leaf, Astragalus, Barberry, Bacopa, Bilberry, Black Cohosh, Black Elderberry, Black Pepper (Plant), Blueberry Leaf, Boswellia serata, Chinese Skullcap Root, Chinese Skullcap Leaf, Cinnamon, Coleus Forskohlii, Cordyceps Mushroom, Echinacea Angustifolia, Eleuthero, Feverfew, Ginger, Ginkgo, Golden Oyster Mushroom, Goldenseal Root, Gotu Kola Leaf, Grape Seed, Green Tea, Hawthorn Berries, Holy Basil, Kava Kava, Korean Ginseng, Lemonbalm, Licorice Root, Lion's Mane, Milk Thistle, Mucana Seed, Olive Leaf, Oregano Leaf, Passion Flower, Phellinus Mushroom, Red Clover Blossom & Leaf, Reishi Mushroom, Rhodiola Rosea, Saw Palmetto, Schisandra Berry, St John's Wort, Tagetes Flower, Turkey Tail Mushroom, Turmeric Root and Valerian Root.

Various further ingredients may be included prior to encapsulation depending on the herbal material extracted. These further ingredients may include additional herbal and non-herbal active materials, anti-caking agents, non-extracted other crude herbal materials, diluents, glidants, and flow agents.

After administering the capsule, the phytochemicals and phytonutrients of the freeze dried extract may have a first release profile and have a rapid release while the phytochemicals and phytonutrients of the crude herbal material may have a second release profile and have a delayed release thus providing a “dual stage” effect. The resulting dual stage herbal nutraceutical may be used as or used in the treatment of, for example, strains and sprains, antiseptics, indigestion, gastrointestinal disorders, antacids, antivirals, treating cramps, antidepressants, cold treatments, laxatives, arthritis treatment, diuretics, anti-anxiety, appetite regulation, insomnia, headaches, treating acne, treating dermatitis, treating asthma, treating eczema, premenstrual syndrome, fatigue and chronic fatigue, dietary supplements, anticoagulants, astringents, antifungals, antioxidants and the like.

The following specific examples are provided to afford a better understanding of the present invention to those skilled in the art. It is to be understood that these samples are intended to be illustrative only and are not intended to limit the invention in any way.

### Examples

Samples were prepared in which Sample A is the invention, Sample B is a slow release crude herbal material and Sample C is a freeze dried extracted herbal powder. Sample A is prepared by extracting turmeric with a 1:1 water to ethanol solution using 25 percent ethanol. Agitation is used to provide an even distribution. After the liquid extract is separated from the crude turmeric, the liquid extract is concentrated to approximately zero percent ethanol. The concentrated liquid turmeric extract is mixed or blended as a 90 percent ethanol-free liquid turmeric extract and 10 percent crude turmeric. To prepare capsules, the following amounts are used:

| | Crude Turmeric | Extracted Turmeric | Black Pepper |
|---|---|---|---|
| Sample A | 180 mg | 300 mg | 10 mg |
| Sample B | 180 mg | 0 mg | 10 mg |
| Sample C | 0 mg | 300 mg | 10 mg |

The capsules are administered to patients with the uptake of turmeric phytochemicals such as curcuminoids of Sample A being comparable to those of Samples B and C.

The foregoing is illustrative of the present invention and is not to be construed as limiting thereof. Although a few exemplary embodiments of this inventio have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific closed embodiments, as well as other embodiments, are intended to be included within the scope of the appended claims. The invention is defined by the following claims, with equivalents of the claims to be included therein.

That which is claimed is:

1. A process for providing a dual stage herbal nutraceutical comprising the steps of:
a) extracting a crude herbal material with a water/alcohol extraction solution to provide an extract separate from crude herbal solids;
b) reducing the alcohol concentration of the extract to provide a low alcohol extract;
c) blending the crude herb material with the low alcohol extract of step b);
d) freeze drying the step c) blend of crude herbal material and low alcohol extract; and
e) encapsulating the freeze dried blend in a plant based capsule.

2. The process of claim 1, wherein the alcohol to water ratio of the extraction solution is from about 20:80 to 30:70 alcohol to water.

3. The process of claim 1, wherein step a) of extracting the crude herbal material includes agitation.

4. The process of claim 1, wherein the step of reducing the alcohol concentration comprises drying at a temperature of 40° C. to 60° C. under vacuum.

5. The process of claim 1, wherein the blend of step c) comprises 60 to 90 percent by weight of the low alcohol extract and 10 to 40 percent by weight of crude herbal material.

6. A dual stage herbal nutraceutical having a freeze dried blend of a low alcohol extract having a first release profile and crude herbal material having a second release profile produced according to claim 1, wherein the low alcohol extract releases rapidly and the crude herbal material release is delayed.

7. A process for providing a dual stage herbal nutraceutical comprising the steps of:
a) extracting a first herbal material and one or more second herbal materials with a water/alcohol extraction solution to provide extracts of the first herbal material and the one or more second herbal extracts separate from herbal solids;
b) mixing the extracts of the first herbal material and the one or more second herbal extracts to provide an extract blend;
c) reducing the alcohol concentration of the extract blend to provide a low alcohol extract blend;
d) blending crude herb of the first herbal material and crude herb of the one or more second herbal materials with the low alcohol extract blend of step c);
e) freeze drying the blend of crude herb materials and low alcohol extract blend to provide a freeze dried blend;
f) encapsulating the freeze dried blend in a plant based capsule.

8. The process of claim 1, wherein the alcohol to water ratio of the extraction solution is from about 20:80 to 30:70 alcohol to water.

9. The process of claim 1, wherein step a) of extracting the first herbal material and one or more second herbal materials includes agitation.

10. The process of claim 1, wherein the step of reducing the alcohol concentration comprises drying at a temperature of 40° C. to 60° C. under vacuum.

11. The process of claim 1, wherein the blend of crude herb materials and low alcohol extract blend of step d) comprises 60 to 90 percent by weight of the low alcohol extract blend and 10 to 40 percent by weight of crude herbal material.

12. A method of treating a medical condition comprising administering the capsule formed by the process of claim 1 to a patent, wherein phytochemicals and phytonutrients in the low alcohol extract are rapidly absorbed and the phytochemicals and phytonutrients of the crude herbal material are slowly absorbed.

13. A method of treating a medical condition comprising administering the capsule formed by the process of claim 7 to a patient, wherein phytochemicals and phytonutrients in the low alcohol extract are rapidly absorbed and the phytochemicals and phytonutrients of the crude herbal material are slowly absorbed.
